(19) United States
(12) Patent Application Publication
(10) Pub. No.: US 2008/0044462 A1
(43) Pub. Date: Feb. 21, 2008

(54) STABILIZED TRANSDERMAL BUPROPION PREPARATIONS

(75) Inventors: Mark W. Trumbore, Westford, MA (US); Roman V. Rariy, Allston, MA (US); Jane C. Hirsh, Wellesley, MA (US); Mark Hirsh, Wellesley, MA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Collegium Pharmaceutical, Inc. Delaware

(21) Appl. No.: 11/697,484

(22) Filed: Apr. 6, 2007

Related U.S. Application Data
(60) Provisional application No. 60/789,635, filed on Apr. 6, 2006.

(51) Int. Cl. A61K 31/205; A61K 9/70; A61K 31/381

(52) U.S. Cl. 424/449; 514/554; 514/438; 514/563

(57) ABSTRACT

Pharmaceutical compositions for transdermal administration containing a fatty acid salt, a dicarboxylic acid salt, an alkyl sulfonic acid salt, an aryl sulfonic acid salt, or an alkyl aryl sulfonic acid salt of an unstable active agent, such as bupropion free base or a derivative of burpropion free base, such as bupropion free base or derivative of bupropion free base, paroxetine, fluvoxamine, fluoxetine, sertraline, venlafaxine, duloxetine, and metabolites and derivatives thereof are described herein. The composition may also contain one or more antioxidants. The compositions can be prepared by forming the bupropion salt followed by addition of the antioxidant. Alternatively, bupropion can be combined first with the antioxidant followed by addition of the acid to form the salt. The compositions can be administered as a gel, cream, lotion, ointment, or patch and typically contain a pharmaceutically acceptable carrier and optionally one or more pharmaceutically acceptable excipients. The compositions described herein are expected to be more stable than bupropion free base and should exhibit excellent dermal penetration.

### CROSS-REFERENCE TO RELATED APPPLICATIONS

This application claims the benefit of the priority of U.S. provisional application 60/789,635, filed Apr. 6, 2006.

### FIELD OF THE INVENTION

The present invention is in the field of transdermal formulations, particularly for the administration of bupropion or a derivative of bupropion.

### BACKGROUND OF THE INVENTION

Bupropion, or 1-(3-chlorophenyl)-2[(1,1,dimethyl ethyl) amino]-1-propanone, is a well-known antidepressant. Its major metabolites, hydroxybupropion (with the hydroxy on the t-butyl group), and two forms hydroxylated on the keto group (threohydrobupropion and erythrohydrobupropion), are also active and have long circulating half lives in the blood stream. Because its structure is different from the major classes of antidepressants, bupropion has several unique uses, including its well known use in smoking cessation. Bupropion has also been used to treat panic disorder, obsessive-compulsive disorder, generalized anxiety disorder, post-traumatic stress disorder, premenstrual dysphoric disorder, and attention deficit/hyperactivity disorder.

Bupropion currently is only approved for oral use in the form of bupropion hydrochloride. Oral use of bupropion has known side effects, including dry mouth, drowsiness, nausea, tremors, dizziness, and seizures. Bupropion is known to lower the seizure threshold, and the frequency of adverse events is dose dependent. (See, for example, Pharmacotherapy 2005 October; 25(10): 1378-82, PubMed abstract).

However, over the 40 years that this compound has been studied, no transdermal dosage form has been successful developed. While adequately stable, the hydrochloride salt has poor transdermal penetration. On the other hand, the free base form of bupropion, which has excellent dermal penetration, is highly unstable in the presence of oxygen. This creates a challenge for those trying to formulate this drug for transdermal delivery. Attempts to stabilize bupropion have been described in U.S. Pat. Nos. 6,312,716 and 6,280,763 to Midha et al. and No. 6,582,737 to Hirsh et al. (using organic antioxidants); U.S. Pat. No. 5,358,970 to Ruff et al. (stablizing bupropion in the form of an organic acid complex using an organic acid having a pH between 0.9 and 4 at an aqueous solution concentration of about 6% w/w and are a solid or liquid at 30° C.); and U.S. Pat. No. 6,153,223 to Apelian et al. (using a pharmaceutically acceptable carrier formed from a dried premixture of a pharmaceutically acceptable carrier and an aqueous solution of an inorganic acid).

U.S. Pat. No. 6,255,502 to Penkler et al. describes the use of bile sales to improve the intestinal permeability of hundreds of drugs, optionally with added cyclodextrins or bile salts to increase permeability within the intestine. However, there is no discussion of stability problems in general or of bupropion in particular. Moreover, none of the formulations described in Penkler are suitable for transdermal delivery across the skin.

Therefore it is an object of the invention to provide transdermal formulations of active agents, particularly unstable active agents such as bupropion, which exhibit improved stability and efficacy, and methods of making and using thereof.

### SUMMARY OF THE INVENTION

Pharmaceutical compositions for transdermal administration containing a fatty acid salt, a dicarboxylic acid salt, an alkyl sulfonic acid salt, aryl sulfonic acid salt, or an alkyl aryl sulfonic acid salt of an active agent, particularly unstable active agents such as bupropion free base or a derivative of bupropion free base, paroxetine, fluvoxamine, fluoxetine, sertraline, venlafaxine, duloxetine, metabolites and derivatives thereof, and combinations thereof, are described herein. The composition may also contain one or more antioxidants. The compositions can be prepared by forming the salt of the unstable active agent followed by addition of the antioxidant. Alternatively, the unstable active agent can be combined first with the antioxidant followed by addition of the acid to form the salt.

The compositions can be administered as a gel, cream, lotion, ointment, aerosol, or patch and typically contain a pharmaceutically acceptable carrier and optionally one or more pharmaceutically acceptable excipients. The compositions described herein are expected to be more stable than free base of the active agent and should exhibit excellent dermal penetration.

### DETAILED DESCRIPTION OF THE INVENTION

I. Definitions

“Bupropion” as used herein, refers to bupropion free base or one or more derivatives or metabolites of bupropion including, but not limited to, hydroxybupropion, threohydrobupropion, and erythrohydrobupropion.

“Antidepressant”, as used herein, refers to a drug or class of drugs used to primarily treat depression. Suitable classes of drugs include, but are not limited to, tricyclic antidepressants, (TCA's), monoamine oxidase inhibitors (MOI's), and serotonin-specific reuptake inhibitors (SSRI's). Examples of these classes of compounds include, but are not limited to, paroxetine, fluvoxamine, fluoxetine, sertraline, venlafaxine, duloxetine, metabolites and derivatives thereof, and combinations thereof. Antidepressants can also be used to treat other disorders including, but not limited to, panic disorder, obsessive-compulsive disorder, generalized anxiety disorder, post-traumatic stress disorder, premenstrual dysphoric disorder, attention deficit/hyperactivity disorder, and smoking.

“Fatty acid”, as used herein, refers to a molecule or moiety having the structure shown below:

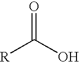

wherein R is a branched or unbranched, saturated or unsaturated aliphatic chain containing 1 to 30 carbon atoms, preferably 5 to 30 carbon atoms, more preferably 8 to 24 carbon atoms, or an anion thereof.

“Dicarboxylic acid”, as used herein, refers to a molecule or moiety having the structure shown below:

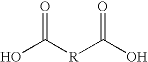

wherein R is a branched or unbranched, saturated or unsaturated aliphatic chain containing 1 to 30 carbon atoms, preferably 5 to 30 carbon atoms, more preferably 8 to 24 carbon atoms, or an anion thereof.

“Alkyl sulfonic acid”, as used herein, refers to a molecule or moiety having the structure shown below:

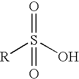

wherein R is a branched or unbranched, saturated or unsaturated aliphatic chain containing 1 to 30 carbon atoms, preferably 5 to 30 carbon atoms, more preferably 8 to 24 carbon atoms, or an anion thereof.

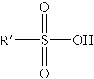

wherein R′ is an aryl group.

“Aryl”, as used herein, includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groupd having heteroatoms in the ring structure may also be referred to as “aryl heterocycles” or “heteroaromatics”. The aromatic ring can be substituted at one or more ring positions with substituents including, but not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocycl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles.

“Alkyl aryl sulfonic acid”, as used herein, refers to a molecule or moiety having the structure shown below:

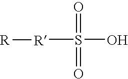

wherein R′ is an aryl group as defined above and R is a branched or unbranched, saturated or unsaturated aliphatic chain containing 1 to 30 carbon atoms, preferably 5 to 30 carbon atoms, more preferably 8 to 24 carbon atoms, or an anion thereof.

II. Compositions

A. Active agent salts

Bupropion, or 1-(3-chlorophenyl)-2[(1,1,dimethyl ethyl) amino]-1-propanone, is a well-known antidepressant of the aminoketon class and is chemically unrelated to tricyclics antidepressants or selective serotonin reuptake inhibitors (SSRIs). It is similar in structure to the stimulant cathinone, anorectic diethylpropion and to phenethylamines in general. Bupropion is both a dopamine reuptake inhibitor and a norepinephrine reuptake inhibitor. It is used as a smoking cessation aid under the brand name Zyban®, and as an antidepressant under the brand name Wellbutrin®, Wellbutrin® SR and Wellbutrin® XL.

The major metabolites of bupropion, hydroxybupropion (with the hydroxy on the t-butyl group), and two forms hydroxylated on the keto group (threohydrobupropion and erythrohydrobupropion), are also active and have long circulating half lives in the blood stream. The structure of bupropion is shown below:

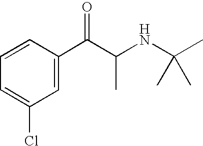

Other active agents can also be used, alone or in combination with bupropion. In one embodiment, the other active agent is an antidepressant. Suitable antidepressants, include, but are not limited to, paroxetine, fluvoxamine, fluoxetine, sertraline, venlafaxine, duloxetine, and metabolites and derivatives thereof. The structure of paroxetine, fluvoxamine, fluoxetine, sertraline, venlafaxine, and duloxetine are shown below:

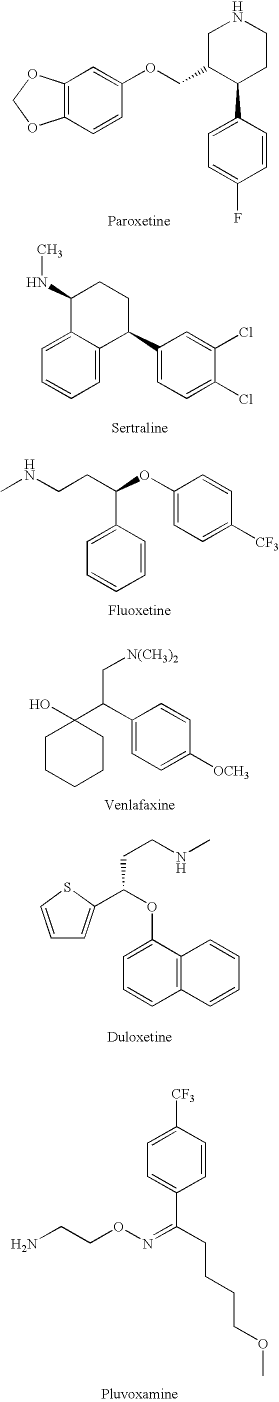

Bupropion/fatty acid or dicarboxylic acid complexes can be prepared by conventional methods known in the art. For example, bupropion base (a liquid at room temperature) is reacted with a fatty acid, either directly or in an organic solvent, such as tetrahydrofuran, ethanol, propanol, or dichloromethane. After the reaction, the bupropion/fatty acid complex can be precipitated from solution or can be recovered by removing the solvent (e.g., evaporation). The resulting complex can be used directly as the active pharmaceutical ingredient (API) in a variety of dosage forms, such as a patch. The molar ratios of the bupropion/carboxylic acid complex may range from about 1.2 to about 0.5 or less. In one embodiment, the ratio of bupropion to carboxylic acid is less than 1 (i.e., carboxyl groups are in molar excess to bupropion base). It is expected that fatty acid salts or dicarboxylic acid salts of other antidepressants, such as paroxetine, fluvoxamine, fluoxetine, sertraline, venlafaxine, duloxetine, and metabolities and derivatives thereof, can be prepared in a similar manner.

The compositions described herein contain a fatty acid salt, dicarboxylic acid salt, alkyl sulfonic acid salt, or an alkyl aryl sulfonic acid salt of an active agent, such as bupropion or an active metabolite of bupropion (“bupropion salt”), paroxetine, fluvoxamine, fluoxetine, sertraline, velafaxine, duloxetine, and metabolites and derivatives thereof. The salts described herein are expected to be more stable than the free base. The salts described herein should exhibit excellent dermal penetration.

Suitable fatty acids which can be used to prepare the salts include, but are not limited to, butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, triacontanoic (melissic) acid, dotriacontanoic (lacceroic) acid, tritriacontanoic (ceromelissic) acid, tetratriacontanoic (geddic) acid, and pentatriacontanoic (ceroplastic) acid.

Suitable dicarboxylic acids include, but are not limited to, succinic, glutaric, adipic, pimelic, suberic, azelaic, sebacic, dodecanedioic, brassylic, thapsic, undercanedioic, tetradecanedioic, pentadecanedioic, hexadecanedioic, octadecanedioic, traumatic acid, itaconic (methylenesuccinic), trans-2-hexenedioic, trans-3-hexenedioic, cis-3-octenedioic, cis-4-octenedioic, and trans-3-octenedioic acid. In one embodiment, a dicarboxylic acid which is monoesterified, such as with an antioxidant, is used to form the salt, provided the esterfied antioxidant is not Vitamin E succinate.

Suitable alkylsulfonic acids include, but are not limited to, decane (capric) sulfonic acid, dodecyl (lauric) sulfonic acid, tetradecanoyl (myristic) sulfonic acid, hexadecanoyl (palmitic) sulfonic acid, heptadecanoyl (margaric) sulfonic acid, octadecanoyl (stearic) sulfonic acid, eicosanoyl (arachidic) sulfonic acid, docosanoyl (behenic) sulfonic acid, tetracosanoyl (lignoceric) sulfonic acid, hexacosanoyl (cerotic) sulfonic acid, heptacosanoyl (carboceric) sulfonic acid, octacosanoyl (montamnic) sulfonic acid, triacontanoyl (melissic) sulfonic acid, dotriacontanoyl (lacceroic) sulfonic acid, tritriacontanoyl (ceromelissic) sulfonic acid, tetratriacontanoyl (geddic) acid, and pentatriacontanoyl (ceroplastic) sulfonic acid.

Suitable alkyl aryl sulfonic acids include, but are not limited to, dodecylbenzene sulfonic acid, tetradecanoylbenzene sulfonic acid, hexadecanoyl-benzene sulfonic acid, heptadecanoylbenzene sulfonic acid, octadecanoylbenzene sulfonic acid, eicosanoylbenzene sulfonic acid, docosanoylbenzene sulfonic acid, tetracosanoylbenzene sulfonic acid, hexacosanoylbenzene sulfonic acid, heptacosanoyl-benzene sulfonic acid, octacosanoylbenzene sulfonic acid, triacontanoylbenzene sulfonic acid, dotriacontanoylbenzene sulfonic acid, tritriacontanoylbenzene sulfonic acid, tetratriacontanoylbenzene sulfonic acid, and pentatriacontanoylbenzene sulfonic acid.

The organic acid is preferably inert (nonreactive), and preferably has minimum detergency and is insoluble in water. The lack of significant detergency is important for protection of the skin during the prolonged exposure required for transdermal delivery. In one embodiment, the fatty acid or dicarboxylic acid has at least 5 carbon atoms, preferably at least 10 carbon atoms, and more preferably at least 12 or 14 carbons.

B. Antioxidants

The compositions optionally contain an antioxidant. Suitable antioxidants include, but are not limited to, tocopherols, such as a Vitamin E, optionally esterfied with a carboxylic acid, such as acetate; derivatives of tocopherols with lower dicarboxylic acids, other than succinic acid; 1-ascorbic palmitate; butylated hydroxy anisole (BHA) and butylated hydroxytoluene (BHT), ascorbic acid, fumaric acid, malic acid, propyl gallate, sodium ascorbate, sodium metabisulfite, ascorbyl palmitate, ascorbyl acetate, ascorbyl phosphate, Vitamin A, folic acid, flavons or flavonoids, carotenoids, carotenes, alpha-Carotene, beta-Carotene, cysteine, pharmaceutically acceptable salts thereof, derivatives thereof, and combinations thereof.

The salts described herein can be combined with an antioxidant. The antioxidant reacts with residual oxygen to prevent oxidation of the active agent. The antioxidant can be added before or after formation of the active agent salt. For example, the anti-oxidant can be added to the free base of the active agent at some time before formation of the salt. A fatty acid or dicarboxylic acid is then added which results in the formation of the salt. Alternatively, the salt is formed first followed by the addition of the antioxidant. In one embodiment, the active agent is bupropion or a derivative or metabolite or bupropion.

In one embodiment, the antioxidant may be covalently coupled to a dicarboxylic acid. For example, one of the carboxylic acid groups can be esterified with the antioxidant. The antioxidant reacts with oxygen to prevent oxidation of the active agent, while the carboxylic acid portion of the molecule forms a salt with the active agent, which should be more stable than the free base and should exhibit excellent dermal penetration.

The antioxidant can also be added as a complex with an ion-exchange resin, also known as a “resinate”, particularly when the antioxidant is an anionic antioxidant.

Complexes are generally prepared by complexing the antioxidant with a pharmaceutically acceptable ion-exchange resin. The complex is formed by reaction of an anionic group on the antioxidant with a cationic functional group on the ion exchange resin.

Ion-exchange resins are water-insoluble, cross-linked polymers containing covalently bound salt forming groups in repeating positions on the polymer chain. The ion-exchange resins suitable for use in these preparations consist of a pharmacologically inert organic or inorganic matrix and contain cationic groups. The organic or inorganic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can also be, e.g., silica gel modified by the addition of ionic groups. The covalently bound salt forming groups may be strongly acidic (e.g., sulfonic acid or sulfuric acid) or weakly acidic (e.g., carboxylic acid). Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975), incorporated by reference herein.

Suitable resin include, but are not limited to, those resins available under the trademark DOWEX®, such as DOWEX® MARATHOS, DOWEX® UPCORE, and DOWEX® SBR. Commercial sources of ion exchange resins that are either pharmaceutically acceptable or may become pharmaceutically acceptable in the future include, but are not limited to, Rohm and Haas, The Dow Chemical Company, and Ion Exchange (India) Ltd. The size of the ion-exchange particles should be less than about 2 millimeter, more preferably below about 1000 micron, more preferably below about 500 micron, and most preferably below about 150 micron.

The antioxidant is bound to the resin by exposure of the resin to the antioxidant in solution via a batch or continuous process (such as in a chromatographic column). The antioxidant-resin complex thus formed is collected by filtration and washed with an appropriate solvent to insure removal of any unbound antioxidant or by-products. Such processes are described in, for example, U.S. Pat. Nos. 4,221,778, 4,894,239, and 4,996,047.

C. Additional Active Agents

The compositions may contain one or more additional active agents which can be administered topically or transdermally. Suitable active agents include, but are not limited to, nicotine and venlafaxine.

D. Carriers, Additives, and Excipients

The compositions described herein can be administered transdermally in a variety of dosage forms including, but not limited to, gels, creams, ointments, aerosols, lotions, and patches. The compositions may contain one or more pharmaceutically acceptable excipients suitable for transdermal formulations. Suitable excipients include, but are not limited to, emulsifiers, diluents, surfactants, solubility enhancers, suspending agents, chelating agents, emollients, humectants, pH modifying agents, lipid bilayer disrupting agents, preservatives, thickening agents, viscosity modifying agents, vitamins and other skin nutrients, and combinations thereof.

Suitable emulsifiers include, but are not limited to, straight chain or branched fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block copolymers, and combinations thereof.

Diluents may be included in the formulations to dissolve, disperse or otherwise incorporate the carrier. Examples of diluents include, but are not limited to, wter, buffered aqueous solutions, organic hydrophilic diluents, such as monovalent alcohols, and low molecular weight glycols and polyols (e.g. propylene glycol, polypropylene glycol, glycerol, butylene glycol).

Suitable surfactants include, but are not limited to, anionic surfactants, non-ionic surfactants, cationic surfactants, and amphoteric surfactants. Examples of anionic surfactants include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethano lamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinnate, disodium lauryl sulfosuccinate, diammonium lauryl sulfosuccinate, tetrasodium N-(1,2-dicarboxyethyl) -N-octadecylsulf-osuccinnate, diamyl ester of sodium sulfosuccinic acid, dihexyl ester of sodium sulfosuccinic acid, dioctyl esters of sodium sulfosuccinic acid, docusate sodium, and combinations thereof.

Examples of nonionic surfactants include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and combinations thereof.

Examples of amphoteric surfactants include, but are not limited to sodium N-dodecyl-alanine, sodium N-lauryl-iminodipropionate, myristoamphoacetate, lauryl betaine, lauryl sulfobetaine, sodium 3-dodecyl-aminopropionate, sodium 3-dodecylaminopropane sulfonate, sodium lauroamphoacetate, cocodimethyl carboxymethyl betaine, cocoamidopropyl baine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyeth-yl betaine, oleamidopropyl betaine, coco dimethyl sulfopropyl betaine, stearyl dimethyl sulfopropyl betaine, lauryl dimethyl sulfoethyl betaine, lauryl bis-2-hydroxyethyl) sulfopropyl betaine, and combinations thereof.

Examples of cationic surfactants include, but are not limited to, behenyl trimethyl ammonium chloride, bis(acyloxyethyl) hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowdimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ehtyl-2-oleyl imidazolinium methyl sulfate, picolin benzyl ammonium chloride, polyquaternium, stearalkonium chloride, sterayl dimethylbenzyl ammonium chloride, stearyl trimethyl ammonium chloride, trimethylglycine, and combinations thereof.

Suitable solubility enhancing agents include solvents such as water; diols, such as propylene glycol and glycerol; mono-alcohols, such as ehtanol, propanol, and higher alcohols; DMSO; dimethylformamide; N,N-dimethylacetamide; 2-pyrrolidone; N-(2-hydroxyethyl) pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones).

Suitable suspending agents include, but are not limited to, alginic acid, bentonite, carbomer, carboxymethylcellulose and salts thereof, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

III. Dosage Forms

The compositions described herein can be administered in a variety of transdermal dosage forms. Suitable dosage forms include, but are not limited to, gels, ointments, lotions, creams, aerosols, and patches. The salts of the active agents described herein are present in the composition in an effective amount. For example, the effective bupropion dosage range is 10-500 mg/day.

A. Transdermal Patch

The bupropion derivatives can be administered in the form of a transdermal patch. Methods for manufacturing transdermal patches are described in U.S. Pat. Nos. 6,312,716; 6,582,737; and 6,280,763. For example, the transdermal patch may contain a base layer (i.e., the layer which will be exposed when the patch is applied). In one embodiment, the base layer has an adhesive peripheral region, and an interior region where the active ingredient is deposited. The active ingredient contains a bupropion salt, optionally in combination with an antioxidant. The base layer is typically impenetrable to the active ingredient as well as water vapor, and is often covered by a foil layer in order to exclude water vapor. The active agent can be applied as a solution or suspension to the base layer. For example, in those embodiments where bupropion is reacted with a fatty acid, an anti-oxidant esterified dicarboxylic acid, an alkyl sulfonic acid or an alky aryl sulfonic acid, the active ingredient can be deposited directly onto the base layer in a single coating process or an injection/deposition process.

In another embodiment, the transdermal patch is a multilayer patch. The active agent, antioxidant and one or more pharmaceutically acceptable excipients can be deposited on a separate layer in a continuous coating operation, cut, and deposited on the base layer via a second layer.

A transdermal patch containing bupropion stearate can be prepared as described in Remington, The Science and Practice of Pharmacy (20th Edition, 2000, Lippincott Williams & Wilikins, pages 917-922). If more complex patch architecture is needed, coating-equilibration processes can be used. In this procedure, a drug free pressure sensitive adhesive skin contact layer can be manufactured by coating onto the release liner. Drug free layers can be applied onto pressure sensitive adhesive skin contact layers in the following order: a non-adhesive acceptor layer followed by a pressure sensitive adhesive anchor layer. Bupropion stearate-containing solution can be applied onto the acceptor layer and the resultant patch will be laminated.

B. Topical Pharmaceutical Vehicles

The compositions described herein can be formulated for administration as gels, creams, lotions, aerosols, or ointments. Creams, lotions, and ointments typically contain one or more excipients such as emulsifiers, surfactants, thickening agents, and combinations thereof. An emulsifier is desirable to help promote efficient release of the actives from the formulation. A wide variety of surfactants are potentially useful. Useful nonionic surfactants include Oleth-10 (polyoxyethylene (10) oleyl ether) in a range from about 1% to about 15%, and presently preferred at about 2% to 7%. Those skilled in the art would be able to test other surfactants, beginning with those having similar HLB, in order to arrive to stable formulations. Mixtures of surfactants can be used to optimize the properties of the formulation.

Any pharmaceutically or cosmetically acceptable thickener suitable for thickening hydrocarbon, silicone or vegetable oils may be used in the formulations. The thickeners modify the rheology of the formulations in order to establish the proper balance between activity and, application and post application physical behavior. Examples of thickeners include colloidal silicas and starches. An example of a preferred thickener is colloidal silica. The thickener is used in a concentration range of between 1.0% to about 5.0%, more preferably in a range of between 1.0% and 2.5%. Those skilled in the art would be able to test other thickeners in order to prepare stable formulations. Mixtures of thickeners can be used to optimize the properties of the formulation.

The formulation may also contain any of a variety of conventional additives and excipients. These can include, without limitation, viscosifiers, additional occluding agent, fragrances, deodorants, colorants, preservatives, vitamins and other skin nutrients, antioxidants, and other stabilizing agents. The various components described above can be collected and provided as a kit.

III. Methods of Use

The compositions can be used to administer an effective amount of bupropion for the treatment of panic disorder, obsessive-compulsive disorder, generalized anxiety disorder, post-traumatic stress disorder, premenstrual dysphoric disorder, and attention deficit/hyperactivity disorder, as well as to aid in the cessation of smoking. One of ordinary skill in the art can readily determine the amount of bupropion to be administered based on the age and weight of the patient and the disorder to be treated.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Preparation of Bupropion Base

Bupropion base can be prepared as described in U.S. Pat. Nos. 6,280,763, 6,312,716 and 6,582,737. In one embodiment, 1.2 g bupropion HCl is dissolved in 20 ml of distilled water to which 0.1 N NaOH is added until the pH is about 12. The mixture is extracted with 20 ml of diethyl ether followed by centrifugation. The ether phase containing the bupropion base is separated and the remaining aqueous phase is extracted three times with 80 ml diethyl ether. The ether phases are combined and dried (removal of residual water) over 15 g anhydrous K₂CO₃, filtered, and the ether is removed via evaporation. The bupropion base is stored under nitrogen gas in a tight bottle in the dark.

The modified procedure of bupropion base preparation is conducted under an inert atmosphere. In this procedure, concentrated ammonium hydroxide solution is used to convert bupropion HCl into bupropion base, the base is extracted three times with diethyl ether and the combined ether phases are dried over sodium sulfate. The solvent is removed under slightly reduced pressure at a water bath temperature of 30° C. The bupropion base is stored under nitrogen gas in a tight bottle in the dark.

### Example 2

### Instability of Bupropion Base Containing Matrices

Several potential stabilizers of bupropion base were evaluated for their ability to prevent discoloration of bupropion-containing patch prototypes. Patch prototype discoloration was used as an indicator of bupropion base stability. The following stabilizers at 0.003% (w/w) were examined: BHA, Vitamin A palmitate, Ascorbyl palmitate, and Vitamin E succinate. Sodium metabisulfite was examined at 0.001% (w/w). The patch prototypes underwent a three step drying process under the following conditions: 15minutes at 25° C., 15 minutes at 40° C. and 15 minutes at 80° C. The bupropion-containing matrices were sealed into pouches and stored for 72 hours at 60° C. in the oven.

Upon the completion of stability study, the pouches were opened and densitometric reflection color measurements were performed using a Techkon CP 230 color measurement system. It was found that while the placebo matrix was completely colorless, even freshly prepared bupropion base containing matrices showed a slight yellow color. The intensity of the color was 4-5 fold higher than that of placebo after 72 hours at 60° C. for all samples tested.

### Example 3

### Proposed Synthesis for Bupropion Stearate

Bupropion base solution in diethyl ether is prepared as described in Example 1. An equimolar solution of stearic acid in diethyl ether will be added dropwise over about 3 hours to a magnetically stirred solution of bupropion base. The mixture will be stirred overnight at ambient temperature. The solvent will be removed under slightly reduced pressure at a water bath temperature of 30° C. The bupropion stearate will be stored in a tight bottle in the dark. The structure of this material will be confirmed by ¹H NMR (CDCl₃), mass spectrometry, elemental analysis and this layer chromatography (TLC).

We claim:

1. A pharmaceutical composition for transdermal administration of an active agent comprising a salt of the active agent selected from the group consisting of fatty acid salts, dicarboxylic acid salts, alkyl sulfate salts, aryl sulfate salts, alkyl aryl sulfonate salts and combinations thereof, in a pharmaceutical carrier suitable for transdermal administration.

2. The composition of claim 1, wherein the active agent is an antidepressant selected from the group consisting of bupropion, paroxetine, fluvoxamine, fluoxetine, sertraline, venlafaxine, duloxetine, derivatives and metabolites thereof, and combinations thereof.

3. The composition of claim 2, wherein the antidepressant is bupropion or a derivative of bupropion.

4. The composition of claim 3, wherein the derivative of bupropion is a metabolite of bupropion selected from the group consisting of hydroxybupropion, threohydrobupropion, and erythrohydrobupropion.

5. The composition of claim 1, further comprising an antioxidant.

6. The composition of claim 5, wherein the antioxidant is selected from the group consisting of tocopherols, optionally esterified with a carboxylic acid; derivatives of tocopherols with lower dicarboxylic acids, other than succinic acid; 1-ascorbic acid palmitate; butylated hydroxy anisole (BHA); butylated hydroxytoluene (BHT); ascorbic acid; fumaric acid; malic acid; propyl gallate; sodium ascorbate; sodium metabisulfite; ascorbyl palmitate; ascorbyl acetate; ascorbyl phosphate; Vitamin A; folic acid; flavones or flavonoids; carotenoids; carotenes; alpha-Carotene; beta-Carotene; cysteine; pharmaceutically acceptable salts thereof; derivatives thereof; and combinations thereof.

7. The composition of claim 1, wherein the salt is a fatty acid salt.

8. The composition of claim 5, wherein the salt is a dicarboxylic acid salt, and wherein one of the carboxylic acid groups of the dicarboxylic acid is esterified with the antioxidant.

9. The composition of claim 1 further comprising at least one other active agent selected from the group consisting of nicotine, venlafaxine, fluoxetine.

10. The composition of claim 1, wherein the fatty acid used to form the salt of bupropion or a derivative of bupropion is selected from the group consisting of butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, triacontanoic (melissic) acid, dotriacontanoic (lacceroic) acid, tritriacontanoic (ceromelissic) acid, tetratriacontanoic (geddic) acid, and pentatriacontanoic (ceroplastic) acid.

11. The composition of claim 1, wherein the dicarboxylic acid used to form the salt of bupropion or a derivative of bupropion is a dicarboxylic acid selected from the group consisting of succinic, glutaric, adipic pimelic, suberic, azelaic, sebacic, dodecanedioic, brassylic, thapsic, undecanedioic, tetradecanedioic, pentadecanedioic, hexadecanedioic, octadecanedioic, traumatic acid, itaconic (methylenesuccinic), trans-2-hexenedioic, trans-3-hexenedioic, cis-3-octenedioic, cis-4-octenedioic, and trans-3-octenedioic acid.

12. The composition of claim 1, wherein the alky sulfonic acid used to form the salt of bupropion or a derivative of bupropion is selected from the group consisting of decane (capric) sulfonic acid, dodecyl (lauric) sulfonic acid, tetradecanoyl (myristic) sulfonic acid, hexadecanoyl (palmitic) sulfonic acid, heptadecanoyl (margaric) sulfonic acid, octadecanoyl (stearic) sulfonic acid, eicosanoyl (arachidic) sulfonic acid, docosanoyl (behenic) sulfonic acid, tetracosanoyl (lignoceric) sulfonic acid, hexacosanoyl (cerotic) sulfonic acid, heptacosanoyl (carboceric) sulfonic acid, octacosanoyl (montanic) sulfonic acid, triacontanoyl (melissic) sulfonic acid, dotriacontanoyl (lacceroic) sulfonic acid, tritriacontanoyl (ceromelissic) sulfonic acid, tetratriacontanoyl (geddic) acid, and pentatriacontanoyl (ceroplastic) sulfonic acid.

13. The composition of claim 1, wherein the alkyl aryl sulfonic acid used to form the salt of bupropion or a derivative of bupropion is selected from the group consisting of dodecylbenzene sulfonic acid, tetradecanoylbenzene sulfonic acid, hexadecanoyl-benzene sulfonic acid, heptadecanoylbenzene sulfonic acid, octadecanoylbenzene sulfonic acid, eicosanoylbenzene sulfonic acid, docosanoylbenzene sulfonic acid, tetracosanoylbenzene sulfonic acid, hexacosanoylbenzene sulfonic acid, heptacosanoyl-benzene sulfonic acid, octacosanoylbenzene sulfonic acid, triacontanoylbenzene sulfonic acid, dotriacontanoylbenzene sulfonic acid, tritriacontanoylbenzene sulfonic acid, tetratriacontanoylbenzene sulfonic acid, and pentatriacontanoylbenzene sulfonic acid.

14. The composition of claim 1, wherein the composition is in the form of a gel, ointment, cream, lotion, aerosol, or patch.

15. A method of making the pharmaceutical composition of claim 1, comprising converting the active agent to a fatty acid, dicarboxylic acid, alkyl sulfate, aryl sulfate, or alkyl aryl sulfonate salt and optionally combining the salt of the active agent with an antioxidant.

16. The method of claim 15, wherein the active agent is an antidepressant selected from the group consisting of bupropion, paroxetine, fluvoxamine, fluoxetine, sertraline, venlafaxine, duloxetine, and derivatives and metabolites thereof.

17. The method of claim 15, wherein the antidepressant is bupropion or a derivative of bupropion.

18. The method of claim 17, wherein the derivative of bupropion is a metabolite of bupropion selected from the group consisting of hydroxybupropion, threohydrobupropion, and erythrohydrobupropion.

19. The method of claim 15, wherein the salt is a fatty acid salt.

20. The method of claim 15, wherein the salt is a dicarboxylic acid salt, and wherein one of the carboxylic acid groups of the dicarboxylic acid is esterified with the antioxidant.

21. The method of claim 15, wherein the antioxidant is selected from the group consisting of tocopherols, optionally esterified with a carboxylic acid; derivatives of tocopherols with lower dicarboxylic acids other than succinic acid; 1-ascorbic palmitate; butylated hydroxy anisole (BHA); butylated hydroxytoluene (BHT); ascorbic acid: fumaric acid; malic acid; propyl gallate; sodium ascorbate; sodium metabisulfite; ascorbyl palmitate; ascorbyl acetate; ascorbyl phosphate; Vitamin A; folic acid; flavones or flavonoids; carotenoids; carotenes; alpha-Carotene; beta-Carotene; cysteine; pharmaceutically acceptable salts thereof; derivatives thereof; and combinations thereof.

22. The method of claim 15 further comprising at least one other active agent selected from the group consisting of nicotine, venlafaxine, and fluoxetine.

23. The method of claim 15, wherein the fatty acid is selected from the group consisting of butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, triacontanoic (melissic) acid, dotriacontanoic (lacceroic) acid, tritriacontanoic (ceromelissic) acid, tetratriacontanoic (geddic) acid, and pentatriacontanoic (ceroplastic) acid.

24. The method of claim 15, wherein the dicarboxylic acid is selected from the group consisting of succinic, glutaric, adipic, pimelic, suberic, azelaic, sebacic, dodecanedioic, brassylic, thapsic, undecanedioic, tetradecanedioic, pentadecanedioic, hexadecanedioic, octadecanedioic, traumatic acid, itaconic (methylenesuccinic) trans-2-hexenedioic, trans-3-hexenedioic, cis-3-octenedioic, cis-4-octenedioic, and trans-3-octenedioic acid.

25. The method of claim 15, wherein the alkyl sulfonic acid is selected from the group consisting of decane (capric) sulfonic acid, dodecyl (lauric) sulfonic acid, tetradecanoyl (myristic) sulfonic acid, hexadecanoyl (palmitic) sulfonic acid, heptadecanoyl (margaric) sulfonic acid, octadecanoyl (stearic) sulfonic acid, eicosanoyl (arachidic) sulfonic acid, docosanoyl (behenic) sulfonic acid, tetracosanoyl (lignoceric) sulfonic acid, hexacosanoyl (cerotic) sulfonic acid, heptacosanoyl (carboceric) sulfonic acid, octacosanoyl (montanic) sulfonic acid, triacontanoyl (melissic) sulfonic acid, dotriacontanoyl (lacceroic) sulfonic acid, tritriacontanoyl (ceromelissic) sulfonic acid, tetratriacontanoyl (geddic) acid, and pentatriacontanoyl (ceroplastic) sulfonic acid.

26. The method of claim 15, wherein the alkyl aryl sulfonic acid is selected from the group consisting of dodecylbenzene sulfonic acid, tetradecanoylbenzene sulfonic acid, hexadecanoyl-benzene sulfonic acid, heptadecanoylbenzene sulfonic acid, octadecanoylbenzene sulfonic acid, eicosanoylbenzene sulfonic acid, docosanoylbenzene sulfonic acid, tetracosanoylbenzene sulfonic acid, hexacosanoylbenzene sulfonic acid, heptacosanoyl-benzene sulfonic acid, octacosanoylbenzene sulfonic acid, triacontanoylbenzene sulfonic acid, dotriacontanoylbenzene sulfonic acid, tritriacontanoylbenzene sulfonic acid, tetratriacontanoylbenzene sulfonic acid, and pentatriacontanoylbenzene sulfonic acid.

27. The method of claim 15, wherein the composition is in the form of a gel, ointment, cream, lotion, aerosol or patch.

28. The method of claim 27, wherein the composition is in the form of a patch.

29. A method for the transdermal delivery of an active agent, comprising administering to a patient in need thereof a composition comprising an effective amount of a carboxylic acid, dicarboxylic acid, alkyl sulfonic acid, aryl sulfonic acid, or alkyl aryl sulfonic acid salt of an active agent.

30. The method of claim 29, wherein the active agent is an antidepressant selected from the group consisting of bupropion, paroxetine, fluvoxamine, fluoxetine, sertraline, venlafaxine, duloxetine, derivatives and metabolites thereof, and combinations thereof.

31. The method of claim 30, wherein the antidepressant is bupropion or a derivative of bupropion.

32. The method of claim 31, wherein the derivative of bupropion is a metabolite of bupropion selected from the group consisting of hydroxybupropion, threohydrobupropion, and erythrohydrobupropion.

33. The method of claim 29, further comprising an antioxidant.

34. The method of claim 33, wherein the antioxidant is selected from the group consisting of tocopherols, optionally esterified with a carboxylic acid; derivatives of tocopherols with lower dicarboxylic acids, other than succinic acid; 1-ascorbic palmitate; butylated hydroxy anisole (BHA); butylated hydroxytoluene (BHT); ascorbic acid; fumaric acid; malic acid; propyl gallate; sodium ascorbate; sodium metabisulfite; ascorbyl palmitate; ascorbyl acetate; ascorbyl phosphate; Vitamin A; folic acid; flavones or flavonoids; carotenoids; carotenes; alpha-Carotene; beta-Carotene; cysteine; pharmaceutically acceptable salts thereof; derivatives thereof; and combinations thereof.

35. The method of claim 29, wherein the salt is a fatty acid salt.

36. The method of claim 33, wherein the salt is a dicarboxylic acid salt, and wherein one of the carboxylic acid groups of the dicarboxylic acid is esterified with the antioxidant.

37. The method of claim 29 further comprising at least one other active agent selected from the group consisting of nicotine, venlafaxine, and fluoxetine.

38. The method claim 35, wherein the fatty acid is selected from the group consisting of butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, triacontanoic (melissic) acid, dotriacontanoic (lacceroic) acid, tritriacontanoic (ceromelissic) acid, tetratriacontanoic (geddic) acid, and pentatriacontanoic (ceroplastic) acid.

39. The method of claim 29, wherein the dicarboxylic acid is selected from the group consisting of succinic, glutaric, adipic, pimelic, suberic, azelaic, sebacic, dodecanedioic, brassylic, thapsic, undecanedioic, tetradecanedioic, pentadecanedioic, hexadecanedioic, octadecanedioic, traumatic acid, itaconic (methylenesuccinic), trans-2-hexenedioic, trans-3-hexenedioic, cis- 3-octenedioic, cis-4-octenedioic, and trans-3-octenedioic acid.

40. The method of claim 29, wherein the alkyl sulfonic acid is selected from the group consisting of decane (capric)) sulfonic acid, dodecyl (lauric) sulfonic acid, tetradecanoyl (myristic) sulfonic acid, hexadecanoyl (palmitic) sulfonic acid, heptadecanoyl (margaric) sulfonic acid, octadecanoyl (stearic) sulfonic acid, eicosanoyl (arachidic) sulfonic acid, docosanoyl (behenic) sulfonic acid, tetracosanoyl (lignoceric) sulfonic acid, hexacosanoyl (cerotic) sulfonic acid, heptacosanoyl (carboceric) sulfonic acid, octacosanoyl (montanic) sulfonic acid, triacontanoyl (melissic) sulfonic acid, dotriacontanoyl (lacceroic) sulfonic acid, tritriacontanoyl (ceromelissic) sulfonic acid, tetratriacontanoyl (geddic) acid, and pentatriacontanoyl (ceroplastic) sulfonic acid.

41. The method of claim 29, wherein the alky aryl sulfonic acid is selected from the group consisting of dodecylbenzene sulfonic acid, tetradecanoylbenzene sulfonic acid, hexadecanoyl-benzene sulfonic acid, heptadecanoylbenzene sulfonic acid, octadecanoylbenzene sulfonic acid, eicosanoylbenzene sulfonic acid, docosanoylbenzene sulfonic acid, tetracosanoylbenzene sulfonic acid, hexacosanoylbenzene sulfonic acid, heptacosanoyl-benzene sulfonic acid, octacosanoylbenzene sulfonic acid, triacontanoylbenzene sulfonic acid, dotriacontanoylbenzene sulfonic acid, tritriacontanoylbenzene sulfonic acid, tetratriacontanoylbenzene sulfonic acid, and pentatriacontanoylbenzene sulfonic acid.

42. The method of claim 29, wherein the composition is in the form of a gel, ointment, cream, lotion, or patch.

43. The method of claim 29, wherein the composition is administered to treat a disorder selected from the group consisting of panic disorder, obsessive-compulsive disorder, generalized anxiety disorder, post-traumatic stress disorder, premenstrual dysphoric disorder, attention deficit/hyperactivity disorder, and smoking.

44. A compound of Formula I 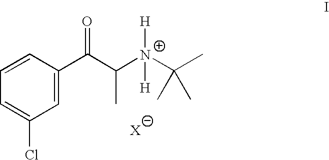
wherein X⁻ is a counterion derived from a compound selected from the group consisting of fatty acids, dicarboxylic acids, alkyl sulfonic acids, aryl sulfonic acids, and alkyl aryl sulfonates.

45. The compound of claim 44, wherein the fatty acid is selected from the group consisting of butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, triacontanoic (melissic) acid, dotriacontanoic (lacceroic) acid, tritriacontanoic (ceromelissic) acid, tetratriacontanoic (geddic) acid, and pentatriacontanoic (ceroplastic) acid.

46. The compound of claim 44, wherein the dicarboxylic is selected from the group consisting of succinic, glutaric, adipic, pimelic, suberic, azelaic, sebacic, dodecanedioic, brassylic, thapsic, undecanedioic, tetradecanedioic, pentadecanedioic, hexadecanedioic, octadecanedioic, traumatic acid, itaconic (methylenesuccinic), trans-2-hexenedioic, trans-3-hexenedioic, cis-3-octenedioic, cid-4-octenedioic, and trans-3-octenedioic acid.

47. The compound of claim 44, wherein the alky sulfonic acid is selected from the group consisting of decane (capric) sulfonic acid, dodecyl (lauric) sulfonic acid, tetradecanoyl (myristic) sulfonic acid, hexadecanoyl (palmitic) sulfonic acid, heptadecanoyl (margaric) sulfonic acid, octadecanoyl (stearic) sulfonic acid, eicosanoyl (arachidic) sulfonic acid, docosanoyl (behenic) sulfonic acid, tetracosanoyl (lignoceric) sulfonic acid, hexacosanoyl (cerotic) sulfonic acid, heptacosanoyl (carboceric) sulfonic acid, octacosanoyl (montanic) sulfonic acid, triacontanoyl (melissic) sulfonic acid, dotriacontanoyl (lacceroic) sulfonic acid, tritriacontanoyl (ceromelissic) sulfonic acid, tetratriacontanoyl (geddic) acid, and pentatriacontanoyl (ceroplastic) sulfonic acid.

48. The compound of claim 44, wherein the alkyl aryl sulfonic acid is selected from the group consisting of dodecylbenzene sulfonic acid, tetradecanoylbenzene sulfonic acid, hexadecanoyl-benzene sulfonic acid, heptadecanoylbenzene sulfonic acid, octadecanoylbenzene sulfonic acid, eicosanoylbenzene sulfonic acid, docosanoylbenzene sulfonic acid, tetracosanoylbenzene sulfonic acid, hexacosanoylbenzene sulfonic acid, heptacosanoyl-benzene sulfonic acid, octacosanoylbenzene sulfonic acid, triacontanoylbenzene sulfonic acid, dotriacontanoylbenzene sulfonic acid, tritriacontanoylbenzene sulfonic acid, tetratriacontanoylbenzene sulfonic acid, and pentatriacontanoylbenzene sulfonic acid.
